(19) United States
(12) Patent Application Publication
(10) Pub. No.: US 2017/0340255 A1
(43) Pub. Date: Nov. 30, 2017

(54) CUPPING DEVICE AND BLOOD ANALYSIS APPARATUS USING THE SAME

(71) Applicants: ELECTRONICS AND TELECOMMUNICATIONS RESEARCH INSTITUTE, Daejeon (KR); Korea Institute of Oriental Medicine, Daejeon (KR)

(72) Inventors: Moon Youn JUNG, Daejeon (KR); Jeong Won PARK, Daejeon (KR); Hyung Ju PARK, Suwon (KR); Dae-Sik LEE, Daejeon (KR); Ju Ah LEE, Daejeon (KR)

(21) Appl. No.: 15/608,926

(22) Filed: May 30, 2017

(30) Foreign Application Priority Data
May 30, 2016 (KR) 10-2016-0066704

(51) Int. Cl. A61B 5/15

(52) U.S. Cl. CPC A61B 5/150099; A61B 5/150755; A61B 5/150251; A61B 5/150259; A61B 5/150343

(57) ABSTRACT

Provided are a cupping device and a blood analysis apparatus using the same. The cupping device includes a cupping cup having a first opening defined in a lower portion thereof, at least one blood collecting container disposed adjacent to an outer surface of the cupping cup and having an accommodation space configured to accommodate blood therein, and a connecting pipe disposed between the cupping cup and the blood collecting container to connect an inner space of the cupping cup to the accommodation space of the blood collecting container.

### CROSS-REFERENCE TO RELATED APPLICATIONS

This U.S. non-provisional patent application claims priority under 35 U.S.C. §119 of Korean Patent Application No. 10-2016-0066704, filed on May 30, 2016, the entire contents of which are hereby incorporated by reference.

### BACKGROUND

The present disclosure herein relates to a cupping device and a blood analysis apparatus using the same.

In general, a cupping treatment is a method of treating a disease in such a manner that a cupping device is closely attached to a skin, and then blood is drawn by using a negative-pressure supply apparatus or applying a physical stimulus such as blood congestion. Also, as the cupping device used for the cupping treatment is widely provided in recent years, the cupping treatment may be easily carried out even in a home. Thus, usefulness thereof has been widely recognized.

### SUMMARY

The present disclosure provides a cupping device easily collecting blood generated during a cupping treatment.

The present disclosure also provides a blood analysis apparatus for analyzing blood collected by a cupping device.

The object of the present invention is not limited to the aforesaid, but other objects not described herein will be clearly understood by those skilled in the art from descriptions below.

An embodiment of the inventive concept provides a cupping device, which is used for a cupping treatment, including: a cupping cup having a first opening defined in a lower portion thereof; at least one blood collecting container disposed adjacent to an outer surface of the cupping cup and having an accommodation space configured to accommodate blood therein; and a connecting pipe disposed between the cupping cup and the blood collecting container to connect an inner space of the cupping cup to the accommodation space of the blood collecting container.

In an embodiment, the blood collecting container may be provided in plurality, and the plurality of blood collecting containers may be spaced apart from each other along the outer surface.

In an embodiment, the blood collecting containers may be disposed to be spaced a predetermined angle with respect to an imaginary first central axis passing through an upper center and a lower center of the cupping cup, and the predetermined angle may be a value obtained by dividing an angle of about 360° by the number of the blood collecting containers.

In an embodiment, an imaginary second central axis passing through an upper center and a lower center of the blood collecting container may make an acute angle with the first central axis.

In an embodiment, the blood collecting container may include: a first side plate disposed to face the outer surface; a second side plate disposed parallel to the first side plate; and a lower plate configured to connect a lower portion of the first side plate to a lower portion of the second side plate. A spaced distance between the first side plate and the outer surface may gradually increase downward.

In an embodiment, the first side plate may have an upper portion contacting the outer surface.

In an embodiment, the blood collecting container may include an upper plate disposed parallel to the lower plate to connect an upper portion of the first side plate to an upper portion of the second side plate, and the upper plate may have a second opening.

In an embodiment, the blood collecting container may further include a closure member configured to cover the second opening.

In an embodiment, the closure member may be made of a rubber material.

In an embodiment, the blood collecting container may be disposed adjacent to a lower end of the cupping cup.

In an embodiment, each of the cupping cup, the blood collecting container, and the connecting pipe may have an inner surface coated with anticoagulant.

In an embodiment of the inventive concept, a blood analysis apparatus includes: a cupping device; a negative pressure supply unit configured to provide a negative pressure to the cupping device; and a blood analysis unit configured to collect and analyze the blood discharged through a skin by the negative pressure. The cupping device includes: a cupping cup having a first opening defined in a lower portion thereof; at least one blood collecting container disposed adjacent to an outer surface of the cupping cup and having an accommodation space configured to accommodate blood therein; and a connecting pipe configured to connect an inner space of the cupping cup to the accommodation space of the blood collecting container.

In an embodiment, the blood analysis unit may include: a pipet configured to collect blood accommodated in the accommodation space of the blood collecting container; and a biosensor configured to analyze the blood collected by the pipet.

In an embodiment, the blood analysis unit may further include a centrifuge configured to centrifugally separate the blood accommodated in the accommodation space of the blood collecting container.

In an embodiment, the centrifuge may have an insertion groove into which the cupping device is inserted.

In an embodiment, the insertion groove may include a first groove into which a portion of the cupping cup is inserted and a second groove into which the blood collecting container is inserted, and the second groove is disposed on a boundary of the first groove.

In an embodiment, the blood collecting container may be provided in plurality, and the plurality of blood collecting containers may be spaced apart from each other along the outer surface.

In an embodiment, the blood collecting containers may be spaced a predetermined angle with respect to an imaginary first central axis passing through an upper center and a lower center of the cupping cup, and the predetermined angle may be a value obtained by dividing an angle of about 360° by the number of the blood collecting containers.

In an embodiment, a second central axis passing through an upper center and a lower center of the blood collecting container may make an acute angle with the first central axis.

In an embodiment, the blood collecting container may include: a first side plate disposed to face the outer surface; a second side plate disposed parallel to the first side plate; and a lower plate configured to connect a lower portion of the first side plate to a lower portion of the second side plate. A spaced distance between the first side plate and the outer surface may gradually increase downward.

Particularities of other embodiments are included in the detailed description and drawings.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a schematic view for explaining a blood analysis apparatus according to embodiments of the inventive concept;

FIG. 2 is a perspective view for explaining a cupping device in FIG. 1;

FIG. 3 is a plan view for explaining the cupping device in FIG. 2;

FIG. 4A is a cross-sectional view taken along line I-I′ of FIG. 2;

FIG. 4B is an enlarged view illustrating a portion A of FIG. 4A;

FIGS. 5A and 5B are schematic views for explaining processes of analyzing blood by using the blood analysis apparatus in FIG. 1;

FIG. 6 is a schematic view for explaining the blood analysis apparatus according to embodiments of the inventive concept;

FIG. 7 is a perspective view for explaining a centrifuge in FIG. 6;

FIG. 8 is a schematic view for explaining a process of separating blood collected by a cupping device by using the centrifuge in FIG. 6; and

FIG. 9 is a view for explaining a state in which blood is separated through the process of FIG. 8.

### DETAILED DESCRIPTION

Advantages and features of the present disclosure, and implementation methods thereof will be clarified through following embodiments described with reference to the accompanying drawings. The present invention may, however, be embodied in many different forms and should not be construed as being limited to the embodiments set forth herein. Rather, these embodiments are provided so that this disclosure will be thorough and complete, and will fully convey the concept of the invention to those skilled in the art. Further, the present invention is only defined by scopes of claims. Like reference numerals refer to like elements throughout.

The embodiment in the detailed description will be described with sectional views and/or plain views as ideal exemplary views of the present invention. In the figures, the dimensions of layers and regions are exaggerated for clarity of illustration. Areas exemplified in the drawings have general properties, and are used to illustrate a specific shape of a semiconductor package region. Thus, this should not be construed as limited to the scope of the present invention. Also, though terms like a first, a second, and a third are used to describe various regions and layers in various embodiments of the inventive concept, the regions and the layers are not limited to these terms. These terms are only used to distinguish one component from another component. An embodiment described and exemplified herein includes a complementary embodiment thereof.

In the following description, the technical terms are used only for explaining a specific exemplary embodiment while not limiting the present invention. The terms of a singular form may include plural forms unless referred to the contrary. The meaning of ‘comprises’ and/or ‘comprising’ specifies a component, a step, an operation and/or an element does not exclude other components, steps, operations and/or elements.

The object of the present invention is not limited to the aforesaid, but other objects not described herein will be clearly understood by those skilled in the art from descriptions below. Also, unless defined apparently in the description, the terms are not ideally or excessively construed as having formal meaning.

Hereinafter, exemplary embodiments will be described in detail with reference to the accompanying drawings.

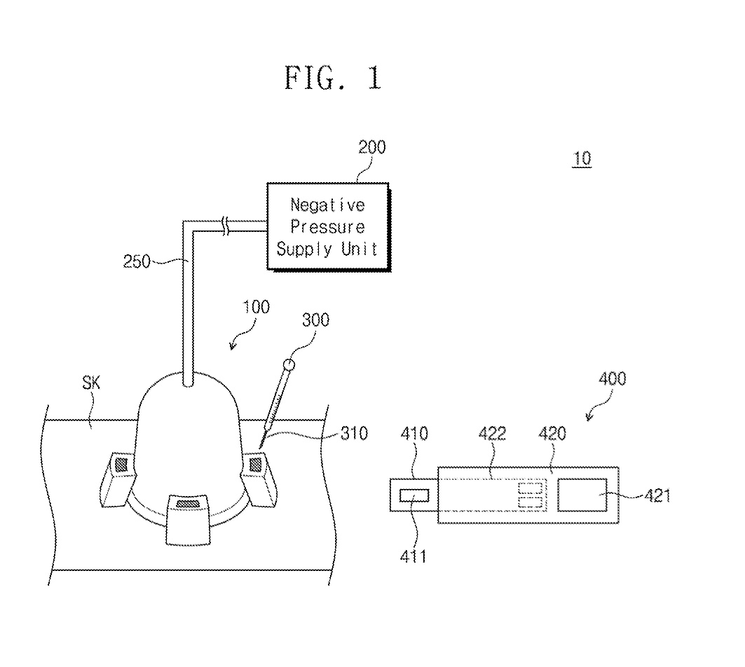

FIG. 1 is a schematic view for explaining a blood analysis apparatus according to embodiments of the inventive concept.

Referring to FIG. 1, a blood analysis apparatus 10 according to embodiments of the inventive concept may analyze blood collected by using a cupping device 100. The blood analysis apparatus 10 may include a cupping device 100, a negative pressure supply unit 200, a pipet 300, and a biosensor 400.

The cupping device 100 may be disposed on a skin SK. In detail, a lower portion of the cupping device 100 may be disposed on the skin SK. The cupping device 100 may be connected to the negative pressure supply unit 200 through a gas exhaust pipe 250.

The negative pressure supply unit 200 may generate a negative pressure. Although the negative pressure supply unit 200 may be a vacuum pump, an embodiment of the inventive concept is not limited thereto. The negative pressure supply unit 200 may provide a negative pressure to the cupping device 100 through the gas exhaust pipe 250. Here, the negative pressure may represent a pressure less than an atmospheric pressure. For example, the negative pressure may be a vacuum pressure. Accordingly, a pressure less than the atmospheric pressure may be formed in an inner space of the cupping device 100. As the negative pressure is formed in the inner space of the cupping device 100, the cupping device 100 may be closely attached to the skin SK, and blood may be drawn through the skin SK on which the acupuncture treatment is provided.

The pipet 300 may be a tool for drawing or dropping a predetermined amount of fluid. For example, the pipet 300 may be a spuit including a microneedle shaped tip 310. However, an embodiment of the inventive concept is not limited the kind of the pipet 300. According to an embodiment, the pipet 300 may draw blood collected by the cupping device 100. The pipet 300 may drop the drawn blood onto the biosensor 400.

The biosensor 400 may measure a constituent contained in the blood. For example, the biosensor 400 may measure blood sugar and C-reactive protein (CRP) contained in the blood. According to embodiments of the inventive concept, the biosensor 400 may be a blood sugar measuring device configured to measure blood sugar. The biosensor 400 may include a blood strip 410 and a blood measuring part 420.

The blood strip 410 may react with the blood to generate an electric signal. Also, the blood strip 410 may have an accommodation groove 411 in which the blood dropped from the pipet 300 is accommodated. The accommodation groove 411 may be disposed adjacent to one end of the blood strip 410.

The blood measuring part 420 may measure a blood constituent by using the electric signal generated from the blood strip 410. The blood measuring part 420 may have a groove 422 into which a portion of the blood strip 410 is inserted. Also, the blood measuring part 420 may include a display portion 421 displaying the measured blood constituent for a user.

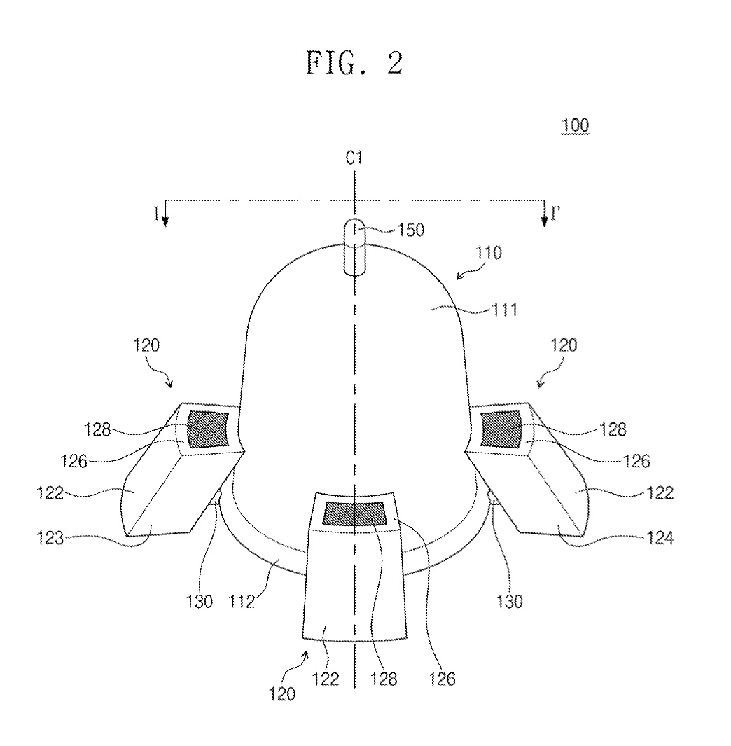

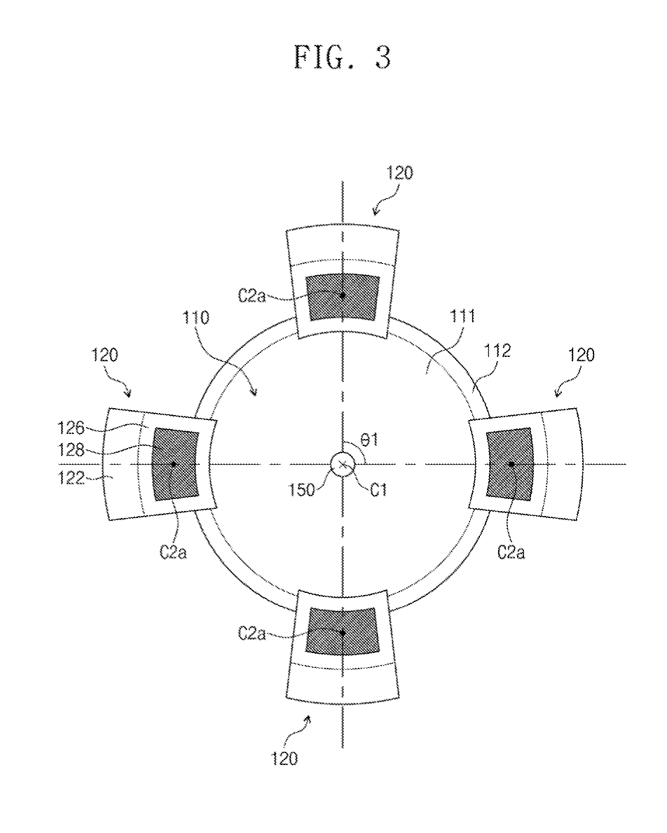

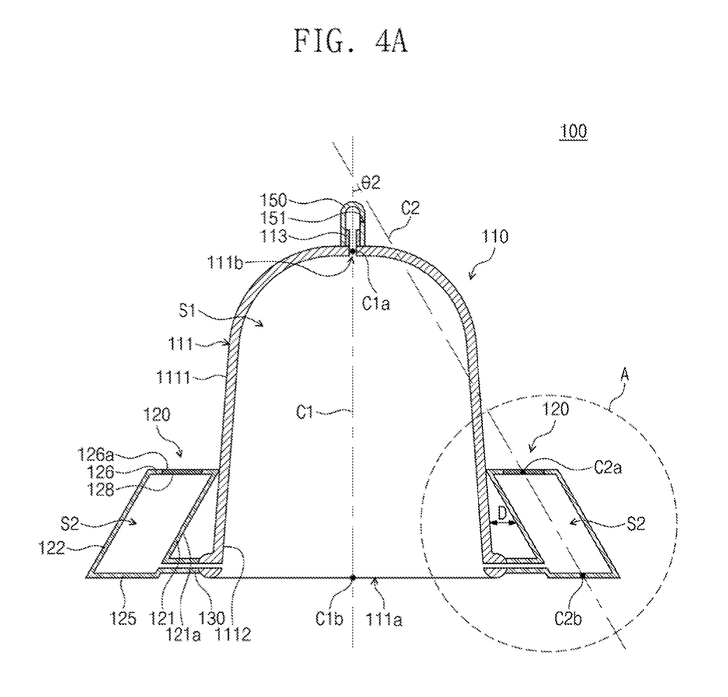

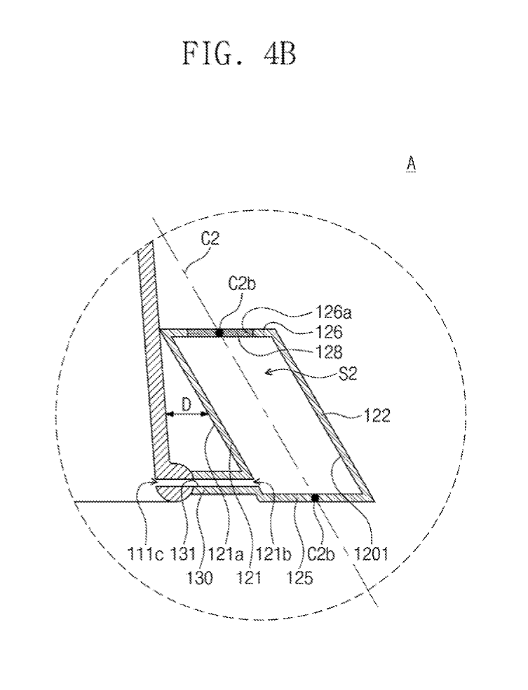

FIG. 2 is a perspective view for explaining the cupping device 100 in FIG. 1. FIG. 3 is a plan view for explaining the cupping device 100 in FIG. 2. FIG. 4A is a cross-sectional view taken along line I-I′ of FIG. 2. FIG. 4B is an enlarged view illustrating a portion A of FIG. 4A.

Referring to FIGS. 1 to 4B, the cupping device 100 according to embodiments of the inventive concept may include a cupping cup 110 having an inner space S1, a blood collecting container 120 having an accommodation space S2 for accommodating blood, and a connecting pipe 130 connecting the inner space S1 of the cupping cup 110 to the accommodation space S2 of the blood collecting container 120. Also, the cupping device 100 may further include a shielding member 150 shielding a gas flow hole 111b of the cupping cup 110, which will be described later.

The cupping cup 110 may be made of a transparent material. For example, the cupping cup 110 may be made of a synthetic resin material that may sustain a negative pressure formed in the inner space S1 and easily secure visibility for checking the inside. Also, the cupping cup 110 may have an imaginary first central axis C1 passing through an upper center C1a and a lower center C1b. The cupping cup 110 may include a body part 111, a flange part 112, and a protruding part 113.

The body part 111 may have a first opening 111a defined in a lower portion thereof. For example, the body part 111 may have a bell shape having an opened lower portion. However, an embodiment of the inventive concept is not limited to the shape of the body part 111. Accordingly, when a negative pressure is formed in the cupping cup 110, the skin SK on which the cupping device 100 is disposed may receive the negative pressure through the first opening 111a. According to an embodiment, although the first opening 111a is provided in a circular shape, an embodiment of the inventive concept is not limited thereto.

The body part 111 may have the gas flow hole 111b connected to the gas exhaust pipe 250. For example, the body part 111 may have the gas flow hole 111b defined in a upper portion thereof. Accordingly, a gas may be flown in and out of the inner space S1 of the cupping cup 110 through the gas flow hole 111b. For example, when a negative pressure is provided in the cupping cup 110, a gas in the cupping cup 110 may be exhausted through the gas flow hole 111b. When a negative pressure is not provided in the cupping cup 110, an external gas may be flown into the cupping cup 110 through the gas flow hole 111b. Also, the gas flow hole 111b may be disposed on the imaginary first central axis C1.

The body part 111 may have at least one discharge hole 111c through which blood is discharged. The discharge hole 111c may be disposed adjacent to a lower end of the body part 111. Also, the discharge hole 111c may be connected to the connecting pipe 130.

The flange part 112 may extend from the lower end of the body part 111 in an outer direction. Accordingly, a contact area between the cupping cup 110 and the skin SK may increase. As the contact area between the cupping cup 110 and the skin SK increases, a force for closely attaching the cupping cup 110 to the skin SK may increase. Although the flange part 112 may have a circular ring shape, an embodiment of the inventive concept is not limited thereto. Here, the outer direction may represent a direction away from the imaginary first central axis C1, and an inner direction may represent a direction toward the imaginary first central axis C1.

The protruding part 113 may have a pipe shape protruding from a boundary of the gas flow hole 111b. Accordingly, the protruding part 113 may have a flow path (not numbered) connected to the gas flow hole 111b therein. According to an embodiment, the protruding part 113 may be disposed on an upper portion of the body part 111.

The blood collecting container 120 may be disposed adjacent to an outer surface 1111 of the body part 111. Also, the blood collecting container 120 may be disposed adjacent to the lower end of the body part 111. The blood collecting container 120 may have an imaginary second central axis C2 passing through an upper center C2a and a lower center C2b. The blood collecting container 120 may have an accommodation space S2 therein. The blood collecting container 120 may have the accommodation space S2 less than the inner space S1 of the cupping cup 110. Also, the blood collecting container 120 may include a container part 121 to 126 and a closure member 128.

The container part 121 to 126 may provide an appearance of the blood collecting container 120. The container part 121 to 126 may have a shape to make an acute angle θ₂ between the second central axis C2 and the first central axis C1. Here, although the acute angle θ₂ between the first and second central axes may be about 15° to about 30°, an embodiment of the inventive concept is not limited thereto. The container part 121 to 126 may include a first side plate 121, a second side plate 122, a third side plate 123, a fourth side plate 124, a lower plate 125, and an upper plate 126.

The first side plate 121 may be disposed adjacent to the outer surface 1111 of the body part 111 closer than the rest side plates 122 to 126. The first side plate 121 may include a first surface 121a facing the outer surface 1111. A spaced distance D between the first surface 121a and the outer surface 1111 may gradually increase downward. According to embodiments, although the spaced distance D may constantly and gradually increase downward, an embodiment of the inventive concept is not limited thereto. Accordingly, a predetermined acute angle (not numbered) may be made between the first surface 121a and the outer surface 1111. Also, the first side plate 121 may include a second surface (not numbered) facing the first surface 121a.

According to an embodiment, since the body part 111 has the bell shape, each of the first surface 121a and the second surface may have a curved surface. Alternatively, in other embodiments, the body part 111 may have a rectangular cylinder shape, and each of the first surface 121a and the second surface may be a flat surface.

The first side plate 121 may have an upper portion contacting the outer surface 1111 of the body part 111. In detail, the first surface 121a may have an upper portion contacting the outer surface 1111 of the body part 111. Also, the first side plate 121 may have an introduction hole 121b defined in a lower portion thereof. The introduction hole 121b may be connected to the connecting pipe 130.

The second side plate 122 may be disposed to face the first side plate 121. For example, the second side plate 122 may be disposed parallel to the first side plate 121. The second side plate 122 may include a third surface (not numbered) facing the second surface of the first side plate 121. The second side plate 122 may include a fourth surface (not numbered) facing the third surface. According to an embodiment, each of the third surface and the fourth surface of the second side plate 122 may be a curved surface.

The third and fourth side plates 123 and 124 may connect both sides of the first side plate 121 to the both sides of the second side plate 122, respectively. According to embodiments, although each of the third and fourth side plates 123 and 124 may have an approximately parallelogram shape, an embodiment of the inventive concept is not limited thereto.

The lower plate 125 may connect a lower portion of each of the first to fourth side plates 121 to 124 to each other. According to embodiments, although the lower plate 125 may have an approximately trapezoidal shape, an embodiment of the inventive concept is not limited thereto.

The upper plate 126 may connect an upper portion of each of the first to fourth side plates 121 to 124 to each other. The upper plate 126 may be disposed parallel to the lower plate 125. The upper plate 126 may have a second opening 126a passing therethrough.

The closure member 128 may cover the second opening 126a. Accordingly, the second opening 126a may be closed. As the second opening 126a is closed, blood in the blood collecting container 120 may be prevented from being evaporated. According to embodiments, the closure member 128 may be made of a rubber material. Accordingly, the tip 310 of the pipet 300 may easily pass through the closure member 128 and inserted into the blood collecting container 120. Also, when the tip 310 is separated from the blood collecting container 120, the penetrated portion of the closure member 128 may be closed again due to an elastic force. Alternatively, in other embodiments, the closure member 128 may be detachably coupled to the upper plate 126 to open and close the second opening 126a.

According to embodiments, the blood collecting container 120 may be provided in plurality. The plurality of blood collecting containers 120 may be spaced along the outer surface 1111 of the body part 111. According to embodiments, the blood collecting containers 120 may be spaced a predetermined angle θ₁ with respect to the imaginary central axis C1. Here, the predetermined angle θ₁ may be a value obtained by dividing an angle of about 360° by the number of the blood collecting containers 120. For example, four blood collecting containers 120 may be spaced an angle of about 90° with respect to the imaginary first central axis C1. Also, three blood collecting containers 120 may be spaced an angle of about 120° with respect to the imaginary first central axis C1. Also, even number blood collecting containers 120 may be symmetric to each other with respect to the imaginary first central axis C1.

The connecting pipe 130 may be disposed between the blood collecting container 120 and the cupping cup 110. The connecting pipe 130 may have a flow path (not numbered), through which blood flows, therein. The connecting pipe 130 has one end connected to a discharge hole 111c of the body part 111 and the other end connected to an introduction hole 121b of the first side plate 121. The connecting pipe 130 may have a straight line shape.

According to embodiments, the cupping cup 110, the blood collecting container 120, and the connecting pipe 130 may be integrally injection-molded. Also, each of inner surfaces 1112, 1201, and 131 of the cupping cup 110, the blood collecting container 120, and the connecting pipe 130 may be coated with anticoagulant. Accordingly, blood generated during the cupping treatment may not be easily coagulated. Here, the anticoagulant may include heparin and the like. According to embodiments, the inner surface 1201 of the blood collecting container 120 may be a second surface of the first side plate 121, a third surface of the second side plate 122, surfaces facing each other of the third and fourth side plates 123 and 124, a top surface of the lower plate 125, and a bottom surface of the upper plate 126.

The shielding member 150 may be disposed on the cupping cup 110. According to an embodiment, although the shielding member 150 may have an elongated cup shape having an opened lower portion, an embodiment of the inventive concept is not limited thereto. The protruding part 113 of the cupping cup 110 may be inserted into the shielding member 150 through an opening of the shielding member 150. Accordingly, the shielding member 150 may be connected to and surround the protruding part 113. As the shielding member 150 is connected to the protruding part 113, the gas flow hole 111b of the body part 111 may be closed.

The shielding member 150 may be connected to the gas exhaust pipe 250. For example, the shielding member 150 may be inserted into the gas exhaust pipe 250. The shielding member 150 may receive a negative pressure from the negative pressure supply unit 200 through the gas exhaust pipe 250. The shielding member 150 may include a cut area 151 in which a portion of a circumference is cut. The cut area 151 may be disposed on a predetermined height upward from a lower end of the shielding member 150. Here, the cut area 151 may be disposed above the protruding part 113 inserted into the shielding member 150.

The cut area 151 may be opened when a negative pressure is provided to the shielding member 150. Accordingly, a gap may be generated at the cut area 151 of the shielding member 150. Also, the negative pressure may be provided into the cupping cup 110 through the gap generated in the shielding member 150. That is, the gas flow hole 111b may be opened.

The shielding member 150 may be made of a material having an elastic force. For example, the shielding member 150 may be made of a rubber material. Accordingly, when the negative pressure provided to the shielding member 150 is stopped, the gap generated in the cut area 151 may be closed by an elastic force.

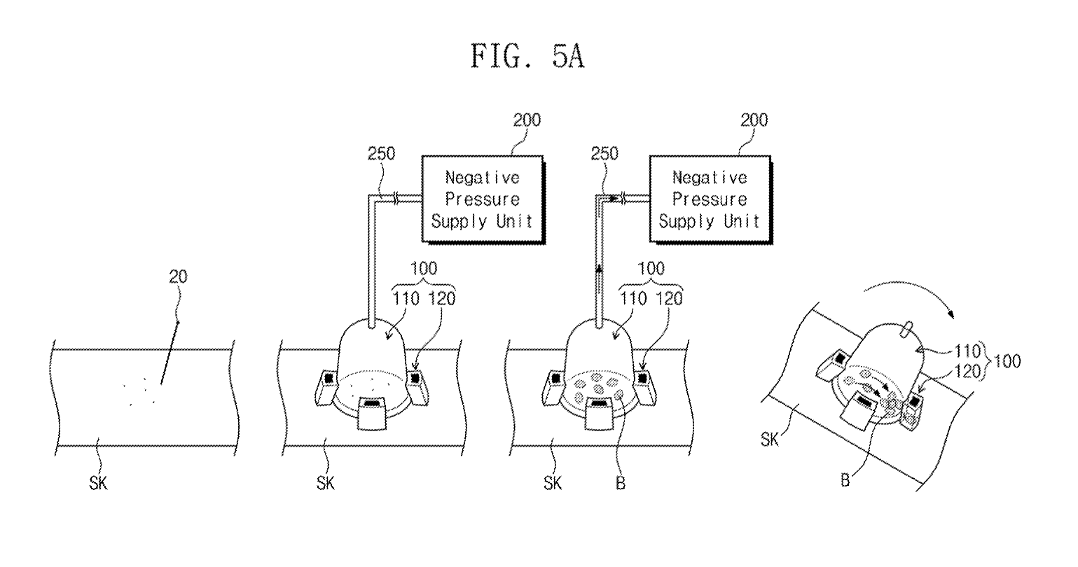

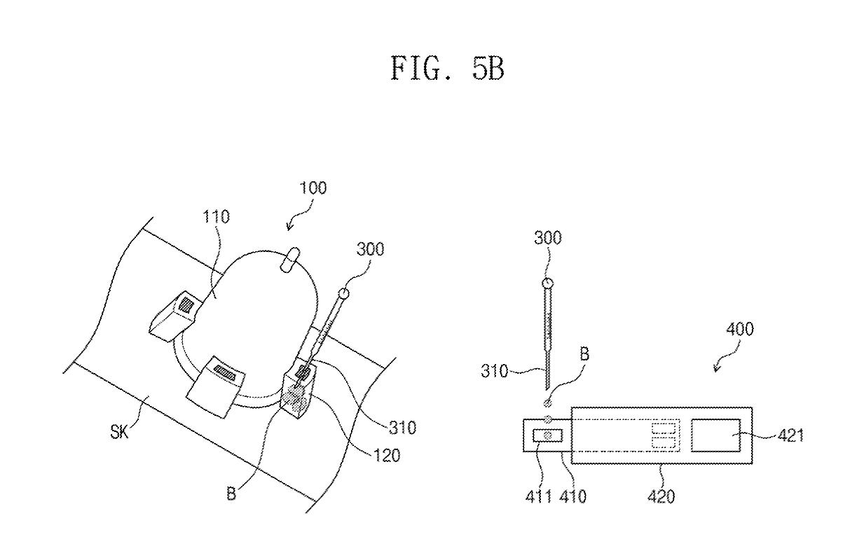

FIGS. 5A and 5B are schematic views for explaining an operation method of the blood analysis apparatus in FIG. 1. FIG. 5A is a view for explaining a process of collecting blood in a blood collecting container, and FIG. 5B is a view for explaining a process of analyzing blood collected in the blood collecting container.

Referring to FIGS. 1 to 5B, a user may perform an acupuncture treatment on the skin SK to be cupping-treated. Here, the acupuncture treatment may represent an action of injecting a needle 20 into the skin SK. The cupping cup 110 may be disposed on the skin SK on which the acupuncture treatment is completed. Here, the skin SK may cover the first opening 111a of the cupping cup 110. After the cupping cup 110 is disposed on the skin SK, the cupping cup 110 may be connected to the negative pressure supply unit 200 through the gas exhaust pipe 250.

The negative pressure supply unit 200 may provide a negative pressure to the cupping device 100 through the gas exhaust pipe 250. When the negative pressure is provided to the cupping device 100, gas in the cupping cup 110 may be discharged through the gap (not shown) generated in the shielding member 150 and the gas flow hole 111b. Accordingly, the negative pressure may be formed in the inner space S1 (refer to FIG. 4A) of the cupping cup 110. The cupping cup 110 may be closely attached to the skin SK by the negative pressure. Accordingly, the first opening 111a may be sealed.

Also, blood B may be drawn from the skin SK by the negative pressure formed in the inner space S1 of the cupping cup 110. The drawn blood B may be flown to the blood collecting container 120 through the connecting pipe 130. Accordingly, the blood may be collected in the accommodation space S2 in the blood collecting container 120. Also, the user may tilt the cupping cup 110 when an amount of the spurted blood B is not enough. Accordingly, the drawn blood B may flow toward any one of the plurality of blood collecting containers 120.

When a predetermined amount of blood B is collected in the blood collecting container 120, the tip 310 of the pipet 300 may pass through the closure member 128 and inserted into the accommodation space S2 (refer to FIG. 4A) of the blood collecting container 120. The pipet 300 may collect the blood B through the tip 310.

The tip 310 of the pipet 300 may be separated from the blood collecting container 120. The pipet 300 may drop the blood B onto the biosensor 400 through the tip 310. In detail, the pipet 300 may drop the blood B onto the accommodation groove 411 of the blood strip 410.

The blood strip 410 may react with the blood accommodated in the accommodation groove 411 to generate an electric signal. The blood measuring part 420 may measure a blood constituent by using the electric signal generated from the blood strip 410. The blood measuring part 420 may display the measured blood constituent on a display portion 421.

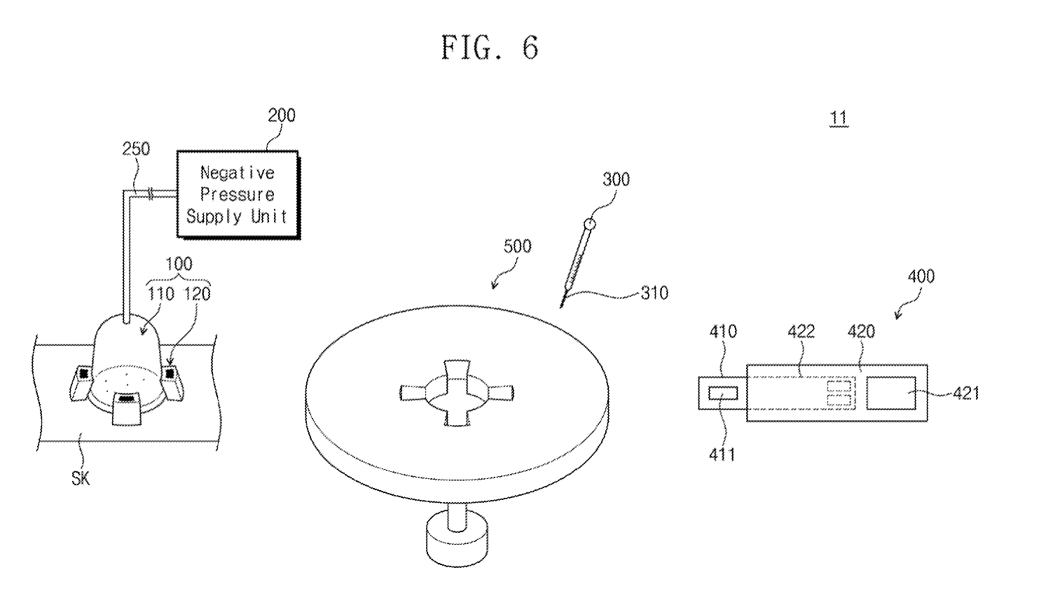

FIG. 6 is a schematic view for explaining the blood analysis apparatus according to embodiments of the inventive concept.

A blood analysis apparatus 11 in FIG. 6 is similar to the blood analysis apparatus 10 described with reference to FIGS. 1 to 4. Thus, detailed description regarding the same component will be omitted or simply described, and different components will be mainly described.

Referring to FIG. 6, the blood analysis apparatus 11 according the embodiments of the inventive concept may include a cupping device 100, a negative pressure supply unit 200, a pipette 300, a centrifuge 500, and a biosensor 400. That is, the blood analysis apparatus 11 according the embodiments of the inventive concept may further include a centrifuge 500 in addition to the blood analysis apparatus 10 in FIG. 1.

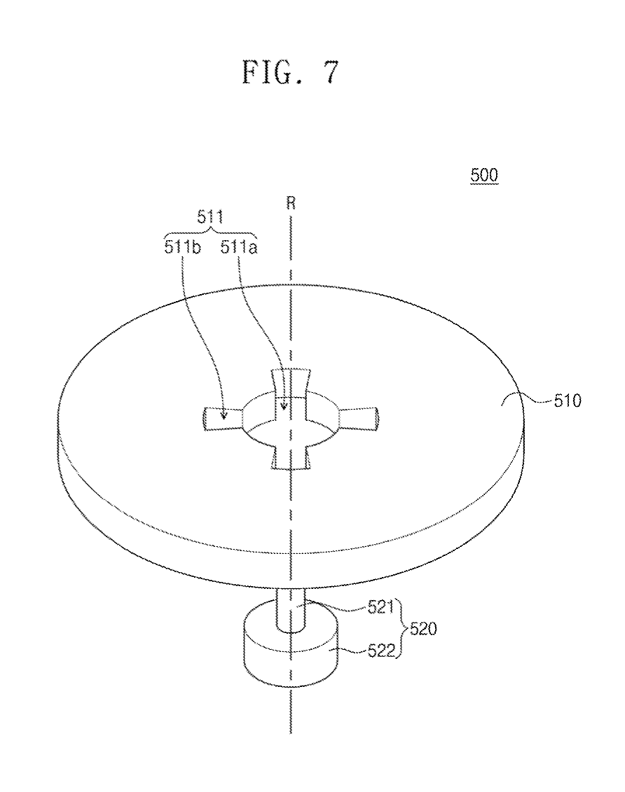

The centrifuge 500 may rotate the cupping device 100. Accordingly, the centrifuge 500 may centrifugally separate blood collected in the blood collecting container 120 of the cupping device 100. This will be described in detail later in FIG. 7.

FIG. 7 is a perspective view for explaining the centrifuge in FIG. 6.

Referring to FIG. 7, the centrifuge 500 may include a rotational plate 510 and a rotational driving unit 520. Also, the centrifuge 500 may include a controller (not shown) controlling the rotational driving unit 520.

Although the rotational plate 510 may have a circular shape, an embodiment of the inventive concept is not limited thereto. The rotational plate 510 may have an imaginary rotational axis R passing through an upper center and a lower center thereof. The rotational plate 510 may an insertion groove 511 into which a portion of the cupping device 100 is inserted. The insertion groove 511 of the rotational plate 510 may include a first groove 511a into which a portion of the cupping cup 110 is inserted and a second groove 511b into which at least a portion of the blood collecting container 120 is inserted.

The first groove 511a may correspond to a lower portion of the cupping cup 110. According to embodiments, the first groove 511a may have a circular shape.

The second groove 511b may be disposed on a boundary of the first groove 511a. The second groove 511b may be connected to first groove 511a. The second groove 511b may correspond to a lower portion of the blood collecting container 120. The number of the second groove 511b may correspond to the number of the blood collecting container 120. According to embodiments, the cupping cup 110 may include four blood collecting containers 120. Accordingly, the centrifuge 500 may include four second grooves 511b.

The plurality of second grooves 511b may be spaced along the boundary of the first groove 511a. In detail, the second grooves 511b may be spaced a predetermined angle with respect to the imaginary rotational axis R. Here, the predetermined angle is a value obtained by dividing an angle of about 360° by the number of the second grooves 511b.

The rotational driving unit 520 may be disposed below the rotational plate 510. The rotational driving unit 520 may include a rotational motor 522 generating a rotational force and a rotational shaft 521 connecting the rotational motor 522 to the rotational plate 510. Also, the rotational shaft 521 may be disposed on the imaginary rotational axis R. For example, the rotational shaft 521 may be elongated along the imaginary rotational axis R. The controller (not shown) may control a rotational speed of the rotational driving unit 520.

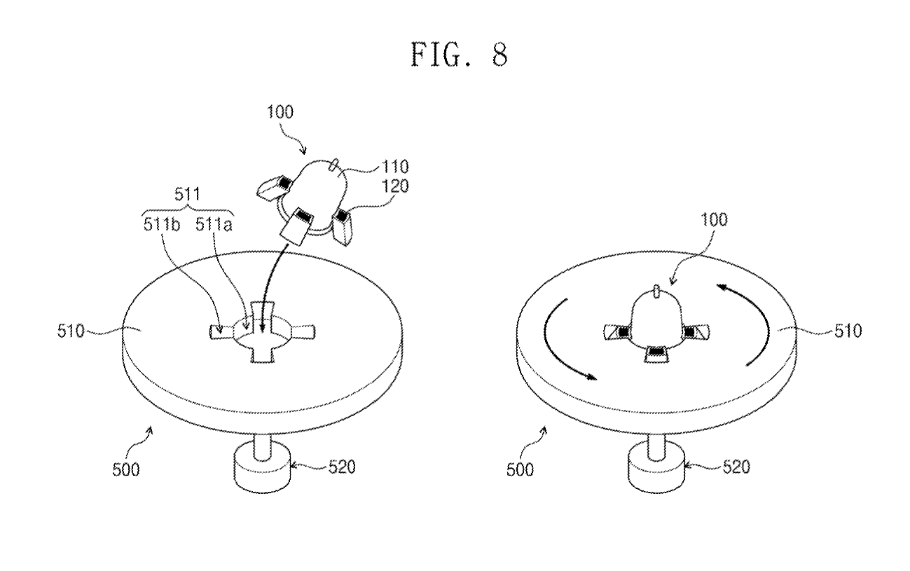

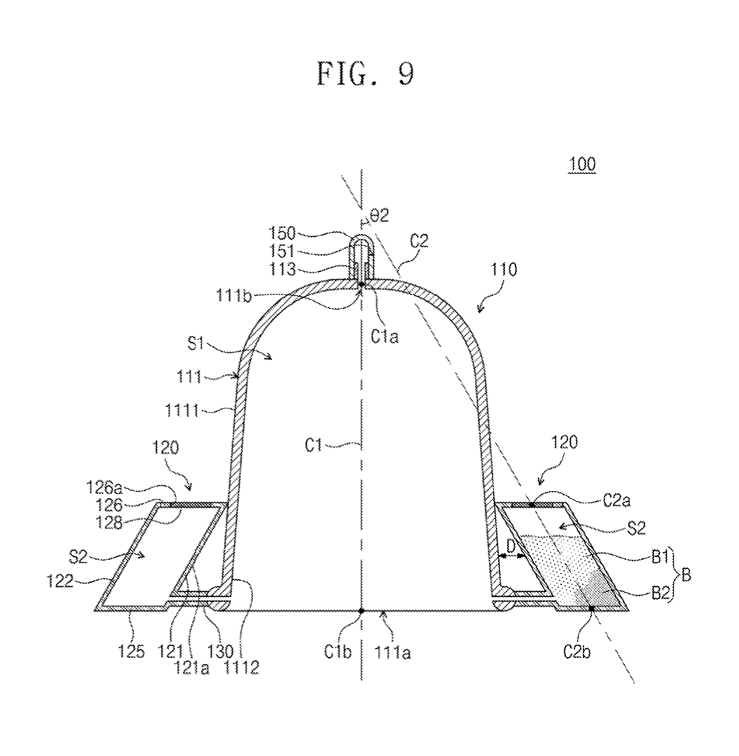

FIG. 8 is a schematic view for explaining a process of separating the blood collected in the cupping device by using the centrifuge in FIG. 6. FIG. 9 is a view for explaining a state in which the blood is separated through the process in FIG. 8. FIG. 9 may correspond to a cross-sectional view taken along line I-I′ of FIG. 4A.

Referring to FIGS. 8 and 9, the cupping device 100 in which blood is collected may be inserted into the insertion groove 511. When the cupping device 100 is inserted into the insertion groove 511, the first central axis C1 (refer to FIG. 4A) of the cupping cup 110 may approximately coincide with the rotational axis R of the rotational plate 510.

The rotational driving unit 520 may rotate the rotational plate 510 clockwise or counterclockwise with respect to the rotational axis R. Accordingly, the cupping device 100 inserted into the insertion groove 511 may rotate. Also, the blood collecting containers 120 may be spaced a predetermined angle along the outer surface of the cupping cup 110 with respect to the first central axis C1 of the cupping cup 110. Accordingly, the cupping device 100 may have a center of gravity approximately disposed on the first central axis C1. Thus, when the cupping device 100 rotates by the centrifuge 500, the cupping device 100 may rotate without vibration. As the cupping device 100 rotates, the blood B collected by the cupping device 100 may be centrifugally separated into blood plasma B1 and a blood cell B2 by a centrifugal force.

The blood cell B2 may move to the outside by the centrifugal force. In general, the centrifugal force may gradually increase away from the rotational axis R. As the second central axis C2 of the blood collecting container 120 and the first central axis C1 make an acute angle θ₂, an edge area at which the lower plate 125 and the second side plate 122 are connected to each other may be the farthest area from the rotational axis R. Accordingly, the largest centrifugal force may act on the edge area at which the lower plate 125 and the second side plate 122 are connected to each other. Thus, most of the blood cells B2 may be disposed on the edge area at which the lower plate 125 and the second side plate 122 are connected to each other That is, most of the blood cells B2 may be disposed on an lower edge of the blood collecting container 120. Accordingly, the pipet 300 may easily collect the blood plasma B1 through the tip 310. The pipet 300 may drop the blood plasma B1 collected from the blood collecting container 120 onto the biosensor 400, and the biosensor 400 may analyze and display a constituent of the blood plasma B1.

According to the embodiments, the cupping device may easily collect the blood generated during the cupping treatment. Also, the blood collected by the cupping device may be analyzed. Thus, the cupping treatment and the blood sugar analysis may be performed at the same time.

The object of the present invention is not limited to the aforesaid, but other objects not described herein will be clearly understood by those skilled in the art from descriptions below.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A cupping device used for a cupping treatment, the cupping device comprising:
a cupping cup having a first opening defined in a lower portion thereof;
at least one blood collecting container disposed adjacent to an outer surface of the cupping cup and having an accommodation space configured to accommodate blood therein; and
a connecting pipe disposed between the cupping cup and the blood collecting container to connect an inner space of the cupping cup to the accommodation space of the blood collecting container.

2. The cupping device of claim 1, wherein the blood collecting container is provided in plurality, and
the plurality of blood collecting containers are spaced apart from each other along the outer surface.

3. The cupping device of claim 2, wherein the blood collecting containers are disposed to be spaced a predetermined angle with respect to an imaginary first central axis passing through an upper center and a lower center of the cupping cup, and
the predetermined angle is a value obtained by dividing an angle of about 360° by the number of the blood collecting containers.

4. The cupping device of claim 3, wherein an imaginary second central axis passing through an upper center and a lower center of the blood collecting container makes an acute angle with the first central axis.

5. The cupping device of claim 1, wherein the blood collecting container comprises:
a first side plate disposed to face the outer surface;
a second side plate disposed parallel to the first side plate; and
a lower plate configured to connect a lower portion of the first side plate to a lower portion of the second side plate,
wherein a spaced distance between the first side plate and the outer surface gradually increases downward.

6. The cupping device of claim 5, wherein the first side plate has an upper portion contacting the outer surface.

7. The cupping device of claim 5, wherein the blood collecting container comprises an upper plate disposed parallel to the lower plate to connect an upper portion of the first side plate to an upper portion of the second side plate, and
the upper plate has a second opening.

8. The cupping device of claim 7, wherein the blood collecting container further comprises a closure member configured to cover the second opening.

9. The cupping device of claim 8, wherein the closure member is made of a rubber material.

10. The cupping device of claim 1, wherein the blood collecting container is disposed adjacent to a lower end of the cupping cup.

11. The cupping device of claim 1, wherein each of the cupping cup, the blood collecting container, and the connecting pipe has an inner surface coated with anti coagulant.

12. A blood analysis apparatus comprising:
a cupping device;
a negative pressure supply unit configured to provide a negative pressure to the cupping device; and
a blood analysis unit configured to collect and analyze the blood discharged through a skin by the negative pressure,
wherein the cupping device comprises:
a cupping cup having a first opening defined in a lower portion thereof;
at least one blood collecting container disposed adjacent to an outer surface of the cupping cup and having an accommodation space configured to accommodate blood therein; and
a connecting pipe configured to connect an inner space of the cupping cup to the accommodation space of the blood collecting container.

13. The blood analysis apparatus of claim 12, wherein the blood analysis unit comprises:
a pipet configured to collect blood accommodated in the accommodation space of the blood collecting container; and
a biosensor configured to analyze the blood collected by the pipet.

14. The blood analysis apparatus of claim 13, wherein the blood analysis unit further comprises a centrifuge configured to centrifugally separate the blood accommodated in the accommodation space of the blood collecting container.

15. The blood analysis apparatus of claim 14, wherein the centrifuge has an insertion groove into which the cupping device is inserted.

16. The blood analysis apparatus of claim 15, wherein the insertion groove comprises a first groove into which a portion of the cupping cup is inserted and a second groove into which the blood collecting container is inserted, and
the second groove is disposed on a boundary of the first groove.

17. The blood analysis apparatus of claim 12, wherein the blood collecting container is provided in plurality, and
the plurality of blood collecting containers are spaced apart from each other along the outer surface.

18. The blood analysis apparatus of claim 17, wherein the blood collecting containers are spaced a predetermined angle with respect to an imaginary first central axis passing through an upper center and a lower center of the cupping cup, and
the predetermined angle is a value obtained by dividing an angle of about 360° by the number of the blood collecting containers.

19. The blood analysis apparatus of claim 18, wherein a second central axis passing through an upper center and a lower center of the blood collecting container makes an acute angle with the first central axis.

20. The blood analysis apparatus of claim 12, wherein the blood collecting container comprises:
a first side plate disposed to face the outer surface;
a second side plate disposed parallel to the first side plate; and
a lower plate configured to connect a lower portion of the first side plate to a lower portion of the second side plate,
wherein a spaced distance between the first side plate and the outer surface gradually increases downward.
